(19) United States
(12) Patent Application Publication
(10) Pub. No.: US 2002/0147368 A1
(43) Pub. Date: Oct. 10, 2002

(54) Branched reaction products of alcohols and aldehydes

(76) Inventors: Wei Li, Mason, OH (US); James A. McCaulley, Ringoes, NJ (US)

Correspondence Address: COGNIS CORPORATION, 2500 RENAISSANCE BLVD., SUITE 200, GULPH MILLS, PA, 19406

(21) Appl. No.: 10/015,524

(22) Filed: Dec. 12, 2001

Related U.S. Application Data
(60) Provisional application No. 60/256,375, filed on Dec. 18, 2000.
(60) Provisional application No. 60/312,731, filed on Aug. 16, 2001.

(51) Int. Cl. C07C 41/00

(52) U.S. Cl. 568/579

(57) ABSTRACT

Compounds of formula III

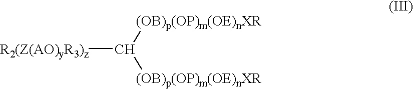

where R₂ is an organic group having from 1 to 36 carbon atoms; Z is —O—, —S—, or —NR₁— where R₁ is hydrogen or a C₁-C₈ alkyl group; AO is ethyleneoxy, propyleneoxy, and/or butyleneoxy; y is a number from 0 to 100; R₃ is a C₁-C₁₀ straight or branched chain alkylene group, or a substituted or unsubstituted aromatic group; z is 0 or 1; p and m are independently numbers of from 0 to 50; n is a number of from 0 to 100; provided that the sum of n, m, and p is at least 1; X is —O—, —S—, or —NR₁—; and R is an organic group having from 4 to 36 carbon atoms; process for their preparation; and aqueous and nonaqueous compositions containing them.

### CROSS REFERENCE TO RELATED APPLICATIONS

This application claims the benefit of copending provisional application serial No. 60/256,375, filed on Dec. 18, 2000, and provisional application serial No. 60/312,731 filed on Aug. 16, 2001; the entire contents of each of which are incorporated herein by reference.

### FIELD OF INVENTION

This invention relates to reaction products useful as low foaming surfactants and as defoaming agents in aqueous and nonaqueous liquid compositions.

### BACKGROUND OF THE INVENTION

Aqueous cleaning compositions exhibit a tendency toward foaming since they contain surface active agents such as soaps, and synthetic detergents. In many instances, such cleaning compositions produce excessive foam and the user must add substances known as anti-foaming agents or defoamers. Some defoamers such as silicones tend to interfere with the function of the cleaning compositions in that unwanted residues are left after the cleaners are wiped off, while others are environmentally unacceptable because they are not biodegradable.

Alkyl polyglycosides are a class of nonionic surfactants that exhibit significantly higher foaming profiles than other nonionic surfactants, such as alcohol ethoxylates. In fact, the foaming tendencies of alkyl polyglycosides more closely resemble those of anionic surfactants, such as alcohol sulfates, than the foaming tendencies of other nonionic surfactants. This higher foaming tendency makes the use of alkyl polyglycosides alone undesirable for many applications, e.g. cleaning-in-place for food processing plants, high pressure spray cleaning, bottle washing, floor cleaners, and automatic dish washing, wherein high levels of foam interfere with the cleaning and rinsing operation and reduce the efficiency of the operation.

Low foam nonionics, such as EO/PO block copolymers, can be used to reduce the foaming properties of alkyl polyglycosides and anionic surfactants, but these materials have undesirable properties, e.g. low biodegradability, relatively high aquatic toxicity, and poor caustic compatibility.

Accordingly, there is a need for the development of defoamers that do not interfere with the cleaning ability of aqueous cleaning compositions and that are biodegradable, exhibit low aquatic toxicity, and good caustic compatibility.

There is also a need for defoamers for nonaqueous compositions.

In addition, there is a continuing need for low foaming surfactants for use in both aqueous and nonaqueous compositions.

### SUMMARY OF THE INVENTION

This invention relates to the reaction products of

A) at least one compound of formula I

RX(EO)ₙ(PO)ₘ(BO)ₚH (I)

wherein R is a substituted or unsubstituted, saturated or unsaturated, organic group having from 4 to 36 carbon atoms; X is —O—, —S—, or —NR₁— where R₁ is hydrogen or a C₁-C₈ alkyl group; n is a number from 0 to 100, e.g., from 1 to 100; m is a number from 0 to 50, e.g. from 1 to 50; and p is a number of from 0 to 50, e.g., from 1 to 50; provided that the sum of n, m, and p is at least 1, and more preferably at least 2; and

B) an aldehyde of formula II

R₂(Z(AO)_{y}R₃)_{z}—CHO (II)

wherein R₂ is a substituted or unsubstituted, saturated or unsaturated, organic group having from 1 to 36 carbon atoms, preferably from 4 to 36 carbon atoms; Z is —O—, —S—, or —NR₁— where R₁ is hydrogen or a C₁-C₈ alkyl group; AO is ethyleneoxy, propyleneoxy, or butyleneoxy, or a random and/or block mixture of two or all three thereof; y=0 to 100; preferably 2 to 100; R₃ is a C₁-C₁₀ straight or branched chain alkylene group, or an aromatic group, e.g. a phenylene group, naphthylene group, and the like, or a substituted aromatic group in which one or more substituents can be present, e.g., C₁-C₄ alkyl groups, halogen groups, —OH groups, C₁-C₄ alkoxy groups, and the like; and z is 0 or 1.

The reaction products of A) and B) will include compounds having the formula III below:

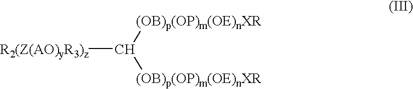

where R₂, Z, AO, y, R₃, z, p, m, n, X, and R have the meanings given above.

### DETAILED DESCRIPTION OF THE INVENTION

Other than in the operating examples, or where otherwise indicated, all numbers expressing quantities of ingredients or reaction conditions used herein are to be understood as modified in all instances by the term “about”.

In the compounds of formulas I and III, it is understood that EO (or OE) stands for the residue of ethylene oxide and PO (or OP) stands for the residue of propylene oxide and BO (or OB) stands for the residue of butylene oxide. Also, in the compounds of formula I, the EO, PO, and BO groups, when present, can be in any order with respect to the RX group, and can be in blocks and/or in random distribution, although the alkoxide groups present are preferably present in the order shown in formulas I and III.

The substituents that can be present on the substituted R groups in formula I can be single or multiple substituents such as one or more halogen substituents, for example Cl, Fl, I, and Br; a sulfur functionality such as a mercaptan or thio group; a nitrogen functionality such as an amine or amide functionality; an alcohol functionality, a silicon functionality, e.g., a siloxane; an ether functionality; or any combination thereof.

As stated above, the R group in formula I can be any substituted or unsubstituted, saturated or unsaturated organic moiety having from 4 to 36 carbon atoms. Thus, when the R group is an aliphatic group, the group can be a linear or branched alkyl group, a linear or branched alkenyl or alkynyl group, a saturated carbocyclic moiety, an unsaturated carbocyclic moiety having one or more multiple bonds, a saturated heterocyclic moiety, an unsaturated heterocyclic moiety having one or more multiple bonds, a substituted linear or branched alkyl group, a substituted linear or branched alkenyl or alkynyl group, a substituted saturated carbocyclic moiety, a substituted unsaturated carbocyclic moiety having one or more multiple bonds, a substituted saturated heterocyclic moiety, and substituted unsaturated heterocyclic moieties having one or more multiple bonds. Examples of the above include but are not limited to an alkyl group having from 4 to 22 carbon atoms, an alkenyl group having from 4 to 22 carbon atoms, and an alkynyl group having from 4 to 22 carbon atoms. R can also be an aromatic group, e.g., phenyl, naphthyl, etc., or an arenyl group. Arenyl groups are alkyl-substituted aromatic radicals having a free valence at an alkyl carbon atom such as a benzylic group. Alkyl groups having from 4 to 12 carbon atoms are preferred, and alkyl groups having from 8 to 10 carbon atoms are most preferred. The degree of ethoxylation is preferably from 2 to 50 with the most preferred being from 4 to about 50 while the degree of propoxylation and butoxylation can vary from 0 to about 50, e.g. from 1 to 10. The degree of propoxylation and/or butoxylation will be determined by the desired degree of solubility or miscibility in aqueous and/or nonaqueous compositions. The solubility and miscibility will ultimately be determined by such factors as the number of carbon atoms in R and the relative amounts of EO, PO, and BO therein, as well as these same factors with respect to the aldehyde of formula II.

The reaction between components A) and B) can be carried out in an inert hydrocarbon solvent at a temperature in the range of from about 20 to 125° C., preferably at a temperature of less than 100° C., e.g. from 20 to 80° C., more preferably from 25 to 65° C. An inert atmosphere such as a nitrogen atmosphere is preferred. The reaction proceeds well in the presence of an acidic catalyst, e.g. paratoluene-sulfonic acid. Other liquid acidic catalysts can also be employed, such as HNO₃, H₂SO₄, H₃PO₄, etc. Also, solid polymeric acidic catalysts, e.g. NAFION® resin (DuPont), AMBERLY® ST 15 (Aldrich chemicals) preferably prewashed with water, can be employed, but the yields of product from such solid polymeric acidic catalysts are not as good as those obtained with p-toluene sulfonic acid. After the reaction has proceeded to completion, usually after 4 to 10 hours, the acid catalyst is neutralized, and the reaction mixture is filtered to produce the filtrate product.

It was found that a competing reaction, i.e. an Aldol condensation between two molecules of component B), followed by the dehydration of the Aldol product to form an alpha, beta-unsaturated aldehyde can occur in parallel with the acetalization reaction. However, selectivity toward the acetalization reaction can be much improved by slow addition of the component B) aldehyde to a mixture of component A) and the acid catalyst, e.g. by dropwise addition of component B) over an extended period of time.

It was also found that when the reaction was carried out at a temperature under 100° C., preferably 80° C. or less, and more preferably 65° C. or less, the competing Aldol condensation reaction was not present or was only present to a limited extent.

The mol ratio of reactants A:B is from 1.75:1 to 3:1, preferably from 2:1 to 2.5:1, and more preferably 2:1 to 2.25:1. Unreacted excess component A) when present is preferably removed from the reaction product, e.g. by thin film evaporation.

The reaction products of the invention can be used as low foaming surfactants in both aqueous and nonaqueous compositions in surfactant-effective amounts, usually from 0.1 to 10% by weight, preferably from 1 to 5% by weight, based on the weight of the composition.

These reaction products can also be used in the above quantities as defoaming agents for aqueous and nonaqueous compositions, and are particularly useful in minimizing or eliminating foaming in aqueous compositions containing high foaming surfactants, such as alkyl polyglycosides and anionic surfactants such as alcohol sulfates.

These reaction products can be used in both aqueous cleaning compositions, emulsion polymer latex compositions such as latex paints, in inks, in adhesives, in metal working compositions, and in other aqueous and nonaqueous compositions in which surfactants and/or defoaming agents are advantageously present.

The reaction products of the invention are biodegradable, contain no organic solvents, and do not adversely affect the detergency of other surfactants that may by present in compositions in which they are used since they are themselves surfactants.

The invention will be illustrated but not limited by the following examples.

### EXAMPLES

### Example 1

Synthesis of Isodecyl Alcohol Ethoxylate Acetal

A mixture of 18.65 g, 44.2 mmol, of PEO(6) isodecyl alcohol; 3.20 g, 20.5 mmol of decyl aldehyde; 20.01 g of heptane as solvent, and 0.095 g, 0.50 mmol, of p-toluenesulfonic acid monohydrate (p-TSOH, as catalyst) were placed in a 50 ml three neck flask, which was equipped with a distillation head apparatus, a nitrogen inlet, and a thermometer. The flask was purged with nitrogen for 10 minutes while stirring, and then heated up to 105° C. for 6.0 hr. After cooling down to room temperature, 0.11 g of 25 wt. % sodium methylate was added to neutralize acid. The mixture was filtered to remove the solid phase. The filtrate was collected as product. IR result showed that aldehyde carboxyl peak (about 1726 cm⁻¹) disappeared. Mw from SEC was 987, which was close to the theoretical value of 1020.

### Example 2

19.993 g (63.9 mmol) of PEO(4) linear C₈-C₁₀ alcohol and 4.528 g (29.0 mmol) of decyl aldehyde were placed in a 100 ml 3-neck round bottom flask equipped with a distillation head apparatus, a nitrogen inlet, a thermometer, an a stir bar. The mixture was heated to 110° C. with stirring and 0.226 g of p-toluenesulfonic acid monohydrate as catalyst was added, and the resulting mixture reacted for 2 hours. The mixture was then cooled to room temperature and 0.26 g (25% w/w) CH₃ONa/CH₃OH added to neutralize the catalyst. 19.089 g of transparent yellow liquid product was obtained, which contained a significant quantity of the dehydrated Aldol condensation by-product.

### Example 3

30.036 g (68.1 mmol) of PEO(6) isodecyl alcohol, 4.942 g (31.6 mmol) of decyl aldehyde, and 0.176 g of p-toluenesulfonic acid monohydrate (0.50% w/w) were added to a 100 ml 3-neck round bottom flask equipped as in Example 2, plus a vacuum pump. The mixture was stirred at 24.5° C., and vacuum (5 Torr) was applied to the reactor to remove water, which shifts the equilibrium toward the acetal product. The reactor contents began boiling vigorously, with the temperature dropping to 15.1° C. The pressure lowered to 1 Torr over the course of about 4 hours. The reactor was then vented with air. 91.397 g of a water-white, crystal clear liquid was present in the reactor. Vacuum at 1 Torr was then applied for another 19 hours and the reactor vented with air. 91.203 g of a slightly hazy liquid was present in the reactor.

Then 0.20 g of 25% w/w CH₃ONa/CH₃OH was added to neutralize the reactor contents. Vacuum was applied for 10 minutes to remove the CH₃OH. The reactor was vented with air, and the reactor contents filtered to give a water-white transparent filtrate product which contained no dehydrated Aldol condensation by-product.

### Example 4

30.106 g of PEO(6) isodecyl alcohol was placed in a 100 ml 3-neck flask equipped as in Example 3. An initial vacuum of 0.40 Torr was applied, causing the liquid to bubble vigorously. The vacuum (0.40-3 Torr) was maintained for 25 minutes. The reactor was vented with air. The reactor contents weighed 29.726 g.

Then 4.876 g of decyl aldehyde was added and vacuum applied for 23 minutes. Vigorous bubbling was again observed. The flask was vented with air and 0.175 g of p-toluenesulfonic acid monohydrate was added and vacuum reapplied. The temperature was raised to 78° C. (bath temperature). The reaction was continued under vacuum for about 4.5 hours. The reaction mixture was then cooled after venting of the vacuum.

Then 0.233 g of 25% w/w CH₃ONa/CH₃OH was added, and vacuum applied for about 30 minutes to remove CH₃OH. The contents of the flask were filtered to give 23.755 g of product, which was a transparent, slightly yellow liquid, containing a small quantity of the dehydrated Aldol condensation by-product.

### Example 5

45.0010 g (102.0 mmol) of PEO(6) isodecyl alcohol, 4.9703 g (31.8 mmol) of decyl aldehyde, and 0.1742 g (0.916 mmol) of p-toluenesulfonic acid were placed in a 100 ml 3-neck flask equipped as in Example 3, and mixed together at ambient temperature. A vacuum of about 5 Torr was applied, and the temperature raised to 55° C. The reaction was carried out at 55° C. and under vacuum for about 4 hours. The reactor contents were then stirred at ambient temperature under vacuum for about 16 hours, and the reactor vented with air. 0.203 g of 25% w/w CH₃ONa/CH₃OH was added to neutralize the reactor contents and a vacuum applied for 20 minutes to remove CH₃OH. The neutralized reactor contents were filtered to give 42.760 g of a water-white crystal clear liquid product, which was free from dehydrated Aldol condensation by-product.

What is claimed is:

1. A compound of formula III 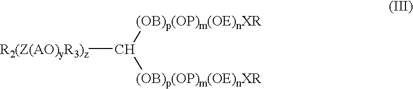
where R₂ is an organic group having from 1 to 36 carbon atoms; Z is —O—, —S—, or —NR_{1— where R}₁ is hydrogen or a C₁-C₈ alkyl group; AO is ethyleneoxy, propyleneoxy, and/or butyleneoxy; y is a number from 0 to 100; R₃ is a C₁-C₁₀ straight or branched chain alkylene group, or a substituted or unsubstituted aromatic group; z is 0 or 1; p and m are independently numbers of from 0 to 50; n is a number of from 0 to 100; provided that the sum of n, m, and p is at least 1; X is —O—, —S—, or —NR₁— where R₁ has the meaning given above; and R is an organic group having from 4 to 36 carbon atoms.

2. The compound of formula III in claim 1 wherein the sum of n, m, and p is at least 2.

3. The compound of formula III in claim 1 wherein Z and X are both —O—.

4. The compound of formula III in claim 1 wherein R is an alkyl group containing from 4 to 22 carbon atoms.

5. The compound of formula III in claim 4 wherein R contains from 4 to 12 carbon atoms.

6. The compound of formula III in claim 1 wherein n=4 to about 50.

7. The compound of formula III in claim 1 wherein OE, OP, and OB when present are in the order shown.

8. The compound of formula III in claim 1 wherein OE, OP, and OB when present are in block and/or random distribution and are in any order with respect to the XR groups.

9. The compound of formula III in claim 1 wherein Z and X are both —O—; R is an alkyl group containing from 4 to 22 carbon atoms; n=4 to about 50; m and p=0; and z=0.

10. The compound of formula III in claim 1 wherein z=0.

11. In an aqueous composition, the improvement wherein a surfactant-effective or defoaming-effective quantity of the compound of claim 1 is present therein.

12. The composition of claim 11 wherein the surfactant-effective or defoaming-effective quantity is from about 0.1 to about 10% by weight.

13. The composition of claim 11 wherein the composition is a latex paint composition.

14. In an aqueous composition containing an alkyl polyglycoside and/or an alcohol sulfate, the improvement wherein a defoaming-effective quantity of the compound of claim 1 is present therein.

15. In a nonaqueous liquid composition, the improvement wherein a surfactant-effective or defoaming-effective quantity of the compound of claim 1 is present therein.

16. The nonaqueous liquid composition of claim 15 wherein the composition is an ink, an adhesive, or a metal working composition.

17. The nonaqueous liquid composition of claim 15 wherein the surfactant-effective quantity is from 0.1 to about 10% by weight.

18. A process for the preparation of a compound of formula III in claim 1 comprising the steps of
A) reacting a compound of formula I
RX(EO)ₙ(PO)ₘ(BO)ₚH (I)
with an aldehyde of formula II
R₂(Z(AO)_{y}R₃)_{z}—CHO (II)
in the presence of a liquid acidic catalyst at a temperature of less than 100° C.; and
B) isolating the compound of formula III from the resulting reaction mixture.

19. The process of claim 18 wherein water is continually removed from the reaction mixture in step A) by azeotropic distillation with an organic solvent.

20. The process of claim 19 wherein the organic solvent is a hydrocarbon solvent.

21. The process of claim 18, wherein in step A) the reaction temperature is less than 80° C.

22. The process of claim 21 wherein the reaction temperature is from about 25 to about 65° C.

23. The process of claim 18 wherein in step A) the acid catalyst is paratoluenesulfonic acid.

24. A reaction product consisting essentially of the product or product mixture obtained by the reaction between the following reactants:
A) at least one compound of formula I:
RX(EO)ₙ(PO)ₘ(BO)ₚH (I)
wherein R is an organic group having from 4 to 36 carbon atoms; X is —O—, —S—, or —NR₁— where R₁ is hydrogen or a C₁-C₈ alkyl group; n is a number of from 0 to 100; p and m are independently numbers of from 0 to 50; provided that the sum of n, m, and p is at least 1; and
B) at least one aldehyde of formula II:
R₂(Z(AO)_{y}R₃)_{z}—CHO (II)
wherein R₂ is an organic group having from 1 to 36 carbon atoms; Z is —O—, —S—, or —NR₁— where R₁ is hydrogen or a C₁-C₈ alkyl group; AO is ethyleneoxy, propyleneoxy, and/or butyleneoxy; y is a number from 0 to 100; R₃ is a C₁-C₁₀ straight or branched chain alkylene group, or a substituted or unsubstituted aromatic group; and z is 0 or 1.

25. The reaction product of claim 24 wherein the reaction is carried out using an acidic catalyst.

26. The reaction product of claim 24 wherein in component A), EO, PO and BO when present are in block and/or random distribution and are in any order with respect to the RX group.

27. The reaction product of claim 24 wherein in component A), EO, PO and BO when present are in the order shown with respect to the RX group.

28. The reaction product of claim 24 wherein in component A), the sum of n, m, and p is at least 2.

29. The reaction product of claim 24 wherein in component A), X is —O—.

30. The reaction product of claim 29 wherein in component A), R is an alkyl group containing from 4 to 22 carbon atoms.

31. The reaction product of claim 30 wherein R contains from 4 to 12 carbon atoms.

32. The reaction product of claim 24 wherein in component A), n=4 to about 50.

33. The reaction product of claim 24 wherein in component A), X is —O—, R is an alkyl group containing from 4 to 22 carbon atoms; n=4 to about 50; and m and p=0.

34. The reaction product of claim 24 wherein in component B), z=0.

35. In an aqueous composition, the improvement wherein a surfactant-effective or defoaming-effective quantity of the reaction product of claim 24 is present therein.

36. In a nonaqueous liquid composition, the improvement wherein a surfactant-effective or defoaming-effective quantity of the reaction product of claim 24 is present therein.
